(19) United States
(12) Patent Application Publication
(10) Pub. No.: US 2007/0161616 A1
(43) Pub. Date: Jul. 12, 2007

(54) Method for treating glaucoma IIB

(76) Inventors: Dilip Wagle, New York, NY (US); Martin Gall, Morristown, NJ (US); Stanley C. Bell, Narberth, PA (US); Edmond J. LaVoie, Princeton Junction, NJ (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/482,666

(22) Filed: Jul. 6, 2006

Related U.S. Application Data
(63) Continuation of application No. 10/036,856, filed on Dec. 31, 2001, now abandoned.
(60) Provisional application No. 60/296,258, filed on Jun. 6, 2001.
(60) Provisional application No. 60/259,428, filed on Dec. 29, 2000.

(51) Int. Cl. A61K 31/541; A61K 31/5377; A61K 31/496; A61K 31/454; A61K 31/427; A61K 31/422; A61K 31/4178

(52) U.S. Cl. 514/210.2; 514/365; 514/374; 514/398; 514/227.5; 514/235.2; 514/235.5; 514/254.02; 514/254.05; 514/326

(57) ABSTRACT

Provided is a method of decreasing intraocular pressure or improving ocular accommodation in an animal, including a human, comprising administering an intraocular pressure decreasing amount or ocular accommodation improving amount of a compound of the formula I or IA, 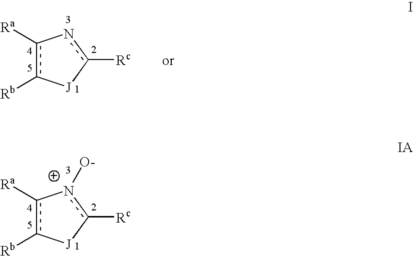
wherein J is oxygen, sulfur, or N—R^{d}.

The present application claims the priority of U.S. Application 60/296,258, filed 6 Jun. 2001, and 60/259,428, filed 29 Dec. 2000.

The present invention relates to methods for treating glaucoma or improving accommodation (i.e. the process by which the eye adjusts for vision at different distances), and to compounds and compositions for use in such treating. In one aspect, the present invention relates to a method of decreasing the intraocular pressure caused by glaucoma.

Diabetes is the major determinant to the development of visual disability and blindness in parts of the world unencumbered by causes related to malnutrition or infectious diseases. Retinopathy is the leading cause of blindness in diabetics and is a progressive, degenerative disease. Of the many risk factors believed to be associated with diabetic retinopathy, the level of glucose in the plasma has been widely investigated. It is well accepted that a lower incidence of retinopathy is associated with decreased plasma levels of glucose.

Ophthalmologic disorders in diabetes include opacification and glaucoma. As the occurrence of these indications is correlated with the persistent hyperglycemia of the disease. Although the incidence of glaucoma is significant in diabetic populations, glaucoma affects a substantial portion of the general aging population as well.

Primary open angle glaucoma occurs in approximately 4% of diabetics compared to 1.8% of the general population. The reasons for the increase in intraocular pressure that is observed in this disorder are not completely understood. The increase in intraocular pressure that characterizes glaucoma is likely caused by an impairment in the drainage of fluid from the eye at the trabecular meshwork since trabeculectomy restores, at least for a period of time, normal intraocular pressures. The origin of this impairment to fluid movement is currently unknown but may be related to a physical obstruction or restriction to movement of proteins that make up a sieving system in the trabecular meshwork. The trabecular meshwork functions as a sieving system that maintains a restricted flow of intraocular fluid from the eye. The result of excess restriction of this flow is a back pressure that causes increased intraocular pressure.

Replacement of the trabecular meshwork (trabeculectomy) remains an established surgical procedure for improving the filtering of intraocular fluid and for overall reduction of intraocular pressure. This remedy is invasive and of limited effectiveness, since pressure elevation frequently recurs after the procedures.

Current chronic pharmaceutical therapies impose a measure of risk on an already medically compromised patient population. The use of topical B-blockers may affect underlying cardiovascular disease, and carbonic anhydrase inhibitors (e.g. Diamox™) may cause metabolic acidosis. The use of pressure-lowering drugs will be affected by the state of renal disease in compromised elderly and diabetic patients. The drawbacks associated with current pharmaceutical therapies highlight an unmet medical need for a chronic pharmaceutical intervention that is distinct in mechanism of action from current therapies.

New strategies for pharmaceutical intervention in the treatment of glaucoma based upon new mechanisms of action need to be identified. In addition, pharmaceutical agents that decrease the intraocular pressure associated with glaucoma are needed. Also, the methods of improving accommodation provided by the invention allow one to avoid costly and burdensome optical solutions, such as the use of separate reading glasses or glasses with bifocal lenses.

### SUMMARY OF THE INVENTION

In one embodiment, the invention relates to a method of treating or ameliorating or preventing glaucoma, decreasing intraocular pressure or improving or ameliorating ocular accommodation in an animal, including a human, comprising administering an intraocular pressure decreasing amount or ocular accommodation improving amount of a compound of the formula I or IA, 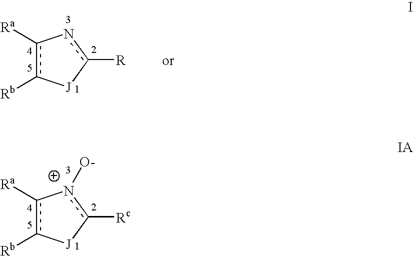
wherein the substituent groups are as defined below.

### DETAILED DESCRIPTION OF THE INVENTION

In accordance with the present invention a method is provided for the treatment of an animal, preferably a mammal, preferably a human with ophthalmologic disorders including glaucoma and reduced accommodation. Briefly the method of the present invention provides for a method of treatment of mammals with glaucoma or reduced accommodation that can be caused by age or certain age-related diseased states such as diabetes. The method provides for administration of classes of inhibitors of advanced glycation. The invention further provides for methods to monitor the improvement in the ocular condition during the course of the administration of compound.

The agents used in the invention are compounds of formulas I or IA, wherein:
a. J is oxygen, sulfur, or N—R^{d};
b. the carbon 2 to nitrogen bond is a double bond except when R^{c} is oxo;
c. the bond between carbons 4 and 5 is a single bond or a double bond (in one embodiment, a single bond);
d. R^{a} and R^{b} are
   1. independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, co-alkylenesulfonic acid, carbamoyl, carboxy, carboxyalkyl (which alkyl can be substituted with alkyloxyimino), cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar {wherein, consistent with the rules of aromaticity, Ar is C₆ or C₁₀ aryl or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring can be fused to a substituted benzene, pyridine, pyrimidine, pyrazine, pyridazine, or (1,2,3)triazine (wherein the ring fusion is at a carbon-carbon double bond of Ar)(or wherein Ar is as above but not heteroaryl fused to pyridine)(in one embodiment, Ar is C₆ or C₁₀ aryl)}, Ar-alkyl, ArO—, ArSO₂—, ArSO—, ArS—, ArSO₂NH—, ArNH, (N—Ar)(N-alkyl)N—, ArC(O)—, ArC(O)NH—, ArNH—C(O)—, and (N—Ar)(N-alkyl)N—C(O)—, or together R^{a} and R^{b} comprise methylenedioxy- (in one embodiment, R^{a} and R^{b} are not acyloxyalkyl, alkenyl, (C₁-C₃)alkylenedioxy or allyl, or together do not comprise methylenedioxy); or
   2. together with their ring carbons form a C₆- or C₁₀-aryl fused ring; or
   3. together with their ring carbons form a C₅-C₇ fused cycloalkyl ring having up to two double bonds including any fused double bond of the containing group, which cycloalkyl ring can be substituted by one or more of the group consisting of alkyl, alkoxycarbonyl, amino, aminocarbonyl, carboxy, fluoro, or oxo (in one embodiment, the ring having no double bonds except any fused double bond of the formula I or IA ring, which cycloalkyl ring can be substituted by one or more of the group consisting of alkyl, amino, aminocarbonyl, carboxy, fluoro, or oxo, where multiple substituents are located on different carbon atoms of the cycloalkyl ring, except in the case of alkyl and fluoro substituents, which can be located on the same or different carbon atoms); or
   4. together with their ring carbons form a fused 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N; or
   5. together with their ring carbons form a fused five to eight membered second heterocycle (in one embodiment, a fused five to six membered second heterocycle), wherein the fused heterocycle consists of ring atoms selected from the group consisting of carbon, nitrogen, oxygen, sulfur, and S(O)ₙ, wherein n is 1 or 2;
e. R^{d} is alkyl, alkenyl, hydrogen, or Ar;
f. R^{c} is
   1. oxo (when Δ^{2,3} is not present), or (when Δ^{2,3} is present) hydrogen, alkyl, alkylthio, hydrogen, mercapto, amino, amino(C₁-C₅)alkyl, or amino(C₆ or C₁₀)aryl, or wherein the amino of the last three groups can be substituted with
      (a) Ar,
      (b) Ar-Z-, Ar-alkyl-Z-, Ar-Z-alkyl, Ar-amino-Z-, Ar-aminoalkyl-Z-, or Ar-oxyalkyl-Z-, wherein Z is a carbonyl or —SO₂—
      (c) formyl or alkanoyl (in one embodiment, R^{c} is according to one of these optionally substituted three groups; in another. J is S or O (in one embodiment S), and R^{c} is hydrogen, oxo, alkyl, amino, amino(C₁-C₅)alkyl or aminophenyl, wherein the amino of the latter three groups can be substituted as outlined here),
   2. —NHC(O)(CH₂)ₙ-D-R^{e}R^{f}, wherein D is oxygen, sulfur or nitrogen, wherein where D is nitrogen n is 0, 1 or 2, but when D is oxygen or sulfur n=1 or 2, and R^{f} is present only when D is nitrogen, wherein
      (a) R^{e} is
         (1) Ar,
         (2) a group of the formula 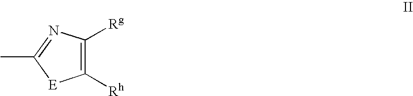
            wherein E is sulfur, oxygen, or N—Rⁱ, and R^{g}, R^{h} and Rⁱ are independently the same as R^{a}, R^{b} and R^{d}, respectively,
         (3) a C₃-C₈ cycloalkyl ring having up to one double bond with the proviso that the carbon linking the cyloalkyl ring to D is saturated, which cycloalkyl ring can be substituted by one or more alkyl-, alkoxycarbonyl-, amino-, aminocarbonyl-, carboxy-, fluoro-, or oxo-substituents;
         (4) a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings;
         (5) hydrogen, (C₂-C₆)hydroxyalkyl, alkanoylalkyl, alkyl, alkoxycarbonylalkyl, alkenyl, carboxyalkyl (which alkyl can be substituted with alkoxyimino), alkoxycarbonyl, a group Ar^{φ} which is C₆- or C₁₀-aryl or a 5- or 6-membered, or 9- or 10-membered heteroaryl (wherein the heteroatom is one oxygen, one sulfur or one nitrogen) or Ar-alkyl; and
      (b) R^{f} is independently hydrogen, (C₂-C₆)hydroxyalkyl, alkanoylalkyl, alkyl, alkoxycarbonylalkyl, alkenyl, carboxyalkyl (which alkyl can be substituted with alkyloxyimino), alkoxycarbonyl, Ar^{φ}, or Ar^{φ}-alkyl;
wherein aryl, Ar, or Ar^{φ} can be substituted with, in addition to any substitutions specifically noted one or more general substituents selected from the group of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, co-alkylenesulfonic acid, alkylthio, allyl, amino, ArC(O)—, ArC(O)NH—, carboxy, carboxyalkyl, cycloalkyl, dialkylamino, halo, trifluoromethyl, hydroxy, (C₂-C₆)hydroxyalkyl, mercapto, nitro, ArO—, Ar—, Ar-alkyl-, sulfamoyl, sulfonic acid, 1-pyrrolidinyl, 4-[C₆ or C₁₀]arylpiperazin-1-yl-, 4-[C₆ or C₁₀]arylpiperidin-1-yl, azetidin-1-yl, morpholin-4-yl, thiomorpholin-4-yl, piperidin-1-yl (in one embodiment, the substituents are selected from the group of aryl preferred general substituents which are: alkyl, amino, dialkylamino, 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl and piperidin-1-yl); and
heterocycles, except those of Ar and Ar^{φ}, can be substituted with in addition to any substitutions specifically noted one or more preferred substituents selected from acylamino, alkanoyl, alkoxy, alkoxycarbonyl, alkoxycarbonylalkyl, alkyl, (C₁ to C₃)alkylenedioxy, alkylamino, alkylsulfonyl, alkylsulfinyl, alkylthio, amino, ArC(O)—, ArO—, Ar—, Ar-alkyl, carboxy, dialkylamino, fluoro, fluoroalkyl, difluoroalkyl, hydroxy, mercapto, oxo, sulfamoyl, trifluoromethyl, 4-[C₆ or C₁₀]arylpiperidin-1-yl and 4-[C₆ or C₁₀]arylpiperazin-1-yl (in one embodiment, the substituents selected from heterocycle preferred general substituents: acylamino, alkanoyl, alkoxy, alkoxycarbonyl, alkoxycarbonylalkyl, alkyl, (C₁ to C₃)alkylenedioxy, alkylamino, alkylsulfonyl, alkylsulfinyl, alkylthio, amino, ArC(O)—, ArO—, Ar—, Ar-alkyl, carboxy, dialkylamino, fluoro, fluoroalkyl, difluoroalkyl, hydroxy, mercapto, oxo, sulfamoyl, trifluoromethyl, 4-[C₆ or C₁₀]arylpiperidin-1-yl and 4-[C₆ or C₁₀]arylpiperazin-1-yl, wherein multiple substituents are located on different atoms of the heterocyclic ring, with the proviso that alkyl, alkoxycarbonyl, and fluoro substituents can be substituted on the same carbon atom of the heterocyclic ring);
   or a pharmaceutically acceptable salt of said compounds,

with the proviso that where the compound of formula I is administered to decrease intraocular pressure at least one compound of formula I administered in effective amount is not a thiazole substituted on a ring carbon sulfonamide (the amide of which can be substituted) that has carbonic anhydrase inhibiting activity.

Primary open angle glaucoma is characterized by an increase in intraocular pressure. The condition of open angle glaucoma is characterized by an increase in the pressure within a person's eye or eyes, called the intraocular pressure. The normal pressure is about 15 mmHg. Elevated pressures of 20-30 mm Hg create a strong risk of damage to the optic nerve and blindness.

Glucose reacts with proteins by a non-enzymatic, post-translational modification process called non-enzymatic glycosylation. The resulting sugar-derived adduct, the advanced glycosylation end product (AGE), matures to a molecular species that is reactive, and can readily bond to amino groups on adjacent proteins, resulting in the formation of AGE cross-links between proteins.

It has now been found that certain compounds that inhibit the formation of such sugar-derived adducts, or in some cases are believed to deactivate such adducts or break resulting crosslinks, can reduce intraocular pressure or ameliorate a trend towards elevated pressure.

Structural matrix proteins isolated from tissues of diabetics and aged individuals are more highly crosslinked than those from nondiabetics or younger individuals and are more resistant to both enzymatic and chemical hydrolysis in vitro. It is this cross-linked state of proteins that is believed to cause stiffness of tissues. The cleavage of AGE cross-links between proteins can provide a mechanism-based therapy for restoration of normal tissue function. An agent that cleaves AGE cross-links between proteins or inhibits their formation can restore more normal sieving function and movement to the trabecular meshwork.

In accordance with the present invention, methods for administering pharmaceutical compositions containing compounds have been developed for the treating glaucoma, intraocular pressure associated with glaucoma, and reduced accommodation. These agents are either substituted thiazole, oxazole, or imidazole agents as shown in the Summary section above.

As is noted in the formula for 1 and IA, the invention includes aromatic thiazole, oxazole, and imidazole analogs, as well as non aromatic analogs thereof such as thiazoline, thiazolidine, oxazoline, oxazolidine, imidazoline, and imidazolidine analogs.

The alkyl, and alkenyl groups referred to above include both C₁ to C₆ linear and branched alkyl and alkenyl groups, unless otherwise noted. Alkoxy groups include linear or branched C₁ to C₆ alkoxy groups, unless otherwise noted.

“Ar” (consistent with the rules governing aromaticity) refers to a C₆ or C₁₀ aryl, or a 5 or 6 membered heteroaryl ring. The heteroaryl ring contains at least one and up to three atoms of N for the 6 membered heteroaryl ring. The 5 membered heteroaryl ring contains; (1) from one to three atoms of N, or (2) one atom of O or S and zero to two atoms of N. Nonlimiting examples of heteroaryl groups include: pyrrolyl, furanyl, thienyl, pyridyl, oxazolyl, pyrazolyl, pyrimidinyl, and pyridazinyl.

“Ar” can be fused to either a benzene, pyridine, pyrimidine, pyridazine, or (1,2,3) triazine ring.

As used herein, C₆ or C₁₀ aryl groups and heteroaryl containing five or six, or nine to ten ring members are monocyclic or bicyclic.

In certain embodiments of the invention, the thiazoles, imidazoles, and oxazoles of the invention contain R^{a} and R^{b} substitutions that together with their ring carbons (the C₄-C₅ carbons of the thiazoles, imidazoles, and oxazoles) form a five to eight membered fused heterocycle (i.e. a bicyclic heterocycle is formed). In these embodiments the fused heterocycle is preferably not aromatic. Particular compounds within these embodiments contain sulfur atoms in the fused heterocycle (the ring fused to the thiazoles, imidazoles, and oxazoles). These sulfur atoms in these particular compounds can exist in various oxidation states, as S(O)ₙ, where n is 0, 1, or 2.

In certain embodiments of the invention, thiazoles, imidazoles, and oxazoles of the invention contain R^{a} and R^{b} substitutions that together with their ring carbons (the C4-C5 carbons of the thiazoles, imidazoles, and oxazoles) form a C5 to C7 cycloalkyl ring having up to double bonds including the C₄-C₅ double bond. In other embodiments a cycloalkyl ring is present when R^{e} is a C3 to C8 cycloalkyl ring. The cycloalkyl groups can be substituted by one or more of the group consisting of alkyl-, alkoxycarbonyl-, amino-, aminocarbonyl-, carboxy-, fluoro-, or oxo-substituents. One of ordinary skill in the art will recognize that where cycloalkyl groups contain double bonds, the sp² hybridized carbon atoms can contain only one substituent (which cannot be amino- or oxo-). Sp³ hybridized carbon atoms in the cycloalkyl ring can be geminally substituted with the exception that (1) two amino groups and (2) one amino and one fluoro group can not be substituted on the same sp³ hybridized carbon atom.

In certain embodiments of the invention, the thiazoles, imidazoles, and oxazoles of the invention contain R^{a} and R^{b} substitutions that together with their ring carbons (the C4-C5 carbons of the thiazoles, imidazoles, and oxazoles) form a five or six membered heteroaryl ring (i.e, a bicyclic aromatic heterocycle is formed). A preferred bicyclic aromatic heterocycle of the invention is a purine analog [J is N—R^{d} and R^{a} and R^{b} together with their ring carbons (the C4 and C5 of the imidazole ring) form a pyrimidine ring].

Aryl, Ar, or Ar^{φ} can be substituted with, in addition to any substitutions specifically noted one or more substituents selected from the group of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl [alkylSO₂—], alkylsulfinyl [alkylSO—], co-alkylenesulfonic acid [-alkylSO₃H where n=1 to 6], alkylthio, allyl, amino, ArC(O)—, ArC(O)NH—, carboxy, carboxyalkyl, cycloalkyl, dialkylamino, halo, trifluoromethyl, hydroxy, (C₂-C₆)hydroxyalkyl, mercapto, nitro, ArO—, Ar—, Ar-alkyl-, sulfamoyl, sulfonic acid, morpholin-4-yl, thiomorpholin-4-yl, piperidin-1-yl, 1-pyrrolidinyl, 4-[C₆ or C₁₀]arylpiperidin-1-yl and 4-[C₆ or C₁₀]arylpiperazin-1-yl.

Heterocycles, except those of Ar and Ar^{φ}, can be substituted with in addition to any substitutions specifically noted one or more substituents selected from acylamino, alkanoyl, alkoxy, alkoxycarbonyl, alkoxycarbonylalkyl, alkyl, (C1 to C3)alkylenedioxy, alkylamino, alkylsulfonyl, alkylsulfinyl, alkylthio, amino, ArC(O)—, ArO—, Ar—, Ar-alkyl, carboxy, dialkylamino, fluoro, fluoroalkyl, difluoroalkyl, hydroxy, mercapto, oxo, sulfamoyl, trifluoromethyl, 4-[C₆ or C₁₀]arylpiperidin-1-yl and 4-[C₆ or C₁₀]arylpiperazin-1-yl, wherein multiple substituents are located on different atoms of the heterocyclic ring, with the proviso that alkyl, alkoxycarbonyl, and fluoro substituents can be substituted on the same carbon atom of the heterocyclic ring. Heterocycles can be substituted with one or more substituents.

The halo atoms can be fluoro, chloro, bromo or iodo. Chloro and fluoro are preferred for aryl substitutions.

In some embodiments of this invention, the compounds of formula (I) can form biologically and pharmaceutically acceptable salts. Useful salt forms include the halides (particularly bromides and chlorides), tosylates, methanesulfonates, brosylates, fumarates, maleates, succinates, acetates, mesitylenesulfonates, and the like. Other related salts can be formed using similarly non-toxic, biologically or pharmaceutically acceptable anions.

Representative, non-limiting examples of compounds of the present invention are:
Thiazole
4,5-Dimethylthiazole
4-Methylthiazole
5-Methylthiazole
4-Methyl-5-(2-hydroxyethyl)thiazole
4-Methyl-5-vinylthiazole
Benzothiazole
2-Aminobenzothiazole
2-Amino-4-chlorobenzothiazole
2-Amino-6-chlorobenzothiazole
2,6-Diamino-benzothiazole
2-Aminothiazole
2,4,5-Trimethylthiazole
2-Amino-5-methylthiazole
2-Amino-4-methylthiazole
2-Acetylthiazole
2-Ethyl-4-methylthiazole
30 Ethyl 2-(Formylamino)-4-thiazoleacetate
2-(Formylamino)-alpha-(methoxyimino)-4-thiazoleacetic acid
2-Amino-4-phenylthiazole hydrochloride monohydrate
2-Isobutylthiazole
2-Methyl-2-thiazoline
2-Methyl-2-oxazoline
2-Oxazolidone
2-Amino-4-thiazoleacetic acid
1-(Thiazolyl)-3-phenyl-urea
1-(Thiazolidinyl)-3-(4-fluorophenyl)-urea
(4-fluorophenyl)thiazolin-2-yl amine
2-(4,6-dimethylpyrimidin-2-ylthio)-N-(1,3-thiazol-2′yl)acetamide, also known as N-(Thiazolyl)-2-(4,6-dimethyl-pyrimidin-2-yl-thio)-acetamide 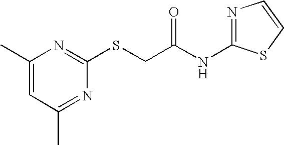
2-(4-propylphenoxy)-N-(thiazol-2-yl)acetamide
2-furyl-N-[4-(6-methylbenzothiazol-2-yl)phenyl]carboxamide
2-(3,5-Dimethylphenoxy)-N-thiazol-2-yl)acetamide
5,5-Dimethyl-2-(2-naphthylamino)-4,5,6-trihydrobenzothiazol-7-one
Imidazole
1-Methylimidazole
1-Ethylimidazole
1-Butylimidazole
1-Vinylimidazole
1-Allylimidazole
1-(Trimethylsilyl)imidazole
1-(3-Aminopropyl)imidazole
1-Benzyl imidazole
1-Phenyl imidazole
1,5-Dicyclohexyl imidazole
1-(p-Toluenesulfonyl)imidazole
N-Benzoyl-imidazole
4-Methyl-imidazole
4′-(Imidazol-1-yl)-acetophenone
4-(Imidazol-1-yl)-phenol
1-(4-Methoxyphenyl)-1H-imidazole
Methyl-4-(1H-imidazol-1yl)benzoate
1-Methylbenzimidazole
   as well as other biologically and pharmaceutically acceptable salts thereof.

Where one or more compounds of formula I are administered to decrease intraocular pressure, at least one compound of formula I administered in effective amount is not a thiazole substituted on a ring carbon sulfonamide (the amide of which can be substituted) that has carbonic anhydrase inhibiting activity. Of course, the composition can include an effective amount of a first agent, as well as a carbonic anhydrase-inhibiting effective amount of another agent, including one of those distinguished above.

Compounds of the formula I can be conveniently prepared by chemical syntheses well-known in the art. Certain of the compounds are well-known and readily available from chemical supply houses or can be prepared by synthetic methods specifically published therefor. For instance, 4,5-Dimethylthiazole, 4-Methylthiazole, 5-Methylthiazole, 4-Methyl-5-thiazoleethanol, 4-Methyl-5-vinylthiazole, Benzothiazole, 2-Aminobenzothiazole, 2-Amino-4-chlorobenzothiazole, 2-Amino-6-chlorobenzothiazole, 2-Aminothiazole, 2,4,5-Trimethylthiazole, 2-Amino-5-methylthiazole, 2-Amino-4-methylthiazole, 2-Acetylthiazole, 2-Ethyl-4-methylthiazole, Ethyl 2-(Formylamino)-4-thiazoleacetate, 2-(Formylamino)-alpha-(methoxyimino)-4-thiazoleacetic acid, 2-amino-4-phenylthiazole hydrochloride monohydrate, 2-Isobutylthiazole, 2-Methyl-2-thiazoline, 2-Methyl-2-oxazoline, 2-Oxazolidone, Thiomorpholine, 2-Amino-4-thiazoleacetic acid, Imidazole, 1-Methylimidazole, 1-Butylimidazole, 1-Vinylimidazole, 1-Allylimidazole, 1-(Trimethylsilyl)imidazole, 1-(3-Aminopropyl)imidazole, 1-Benzyl imidazole, 1-Phenyl imidazole, 1,5-Dicyclohexyl imidazole, 1-(p-Toluenesulfonyl)imidazole, N-Benzoyl-imidazole, 4-Methyl-imidazole, 4′-(Imidazol-1-yl)-acetophenone, 4-(Imidazol-1-yl)-phenol, 1-(4-Methoxyphenyl)-1H-imidazol and 1-Methylbenzimidazole can be obtained from Sigma (St. Louis, Mo.), Aldrich (Milwaukee, Wis.) or Fluka (Milwaukee, Wis.) (all divisions of Sigma-Aldrich Co.). 1-ethylimidazole can be obtained from TCI America (Portland, Oreg.). N-(Thiazolidinyl)-4-fluoroaniline, N-(Thiazolyl)-2-(4,6-dimethyl-pyrimidin-2-yl-thio)-acetamide, N-(Thiazolyl)-2-(4-propylphenoxy)-acetamide, 2-[4-(N-Furoyl)aminophenyl]-6-methylbenzothiazole, N-(Thiazolyl)-2-(3,5-dimethylphenoxy′-acetamide and 2-[(N-(2-Napthalenyl)amino]-[2,3:5,4]-(5,5-dimethyl-cyclohexanonyl)]thiazole can be purchased from MDD, Inc. (Acton, Ontario), a successor to Ortech Corporation.

In one synthetic process to prepare compounds of the general formula I, a thiazole is reacted with an alkyl or acyl halide in the presence of base such as triethylamine, to produce the corresponding alkyl or acyl derivative at the 2 carbon. See, Medici et al., J. Org. Chem. 49: 590-596, 1984. In some cases, a chromatographic step is applied to separate additions at the 2 and 3 positions of the thiazole ring.

In another synthesis of compounds of the formula I wherein R^{c} is amino, nitro-containing analogs of compounds of the invention or precursors thereof are catalytically hydrogenated to the corresponding amino compounds.

2-Amino thiazole compounds wherein (R^{c} is amino) can also be synthesized by reacting thiourea (which can be substituted on at least one amine) with an alpha-halo ketone using the method described in Vogel 's Textbook of Practical Organic Chemistry, 5^{th} Edition, John Wiley & Sons, New York, p. 1153. Such a reaction is exemplified by a synthesis of 2-amino-4-phenylthiazole:

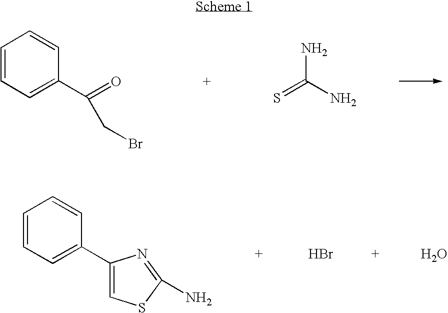

N-aryl imidazoles can be prepared using appropriate aromatic nucleophilic displacement reactions. For example, fluoro phenyl compounds such as 4-fluorobenzoic acid methyl ester can be used to substitute on the N¹ nitrogen of imidazole to make methyl-4-(1H-imidazol-1-yl)benzoate. See, Morgan et al., J. Med. Chem. 33: 1091-1097, 1990.

Amino functions of 2-aminoimidazoles or 2-aminothiazoles can be acylated by dehydration or other methods known in the art.

Substituted oxazoles can be prepared by methods known in the art. For instance, 2-unsubstituted oxazoles can be formed by condensation of formamide with either α-hydroxy or α-haloketones intermediates (H. Bredereck, R. Gommper, H. G. v. Shuh and G. Theilig, in Newer Methods of Preparative Organic Chemistry, Vol. III, ed. W. Foerst, Academic press, New York, 1964, p. 241). The intermediates can cyclize under acid conditions to form the oxazole ring (Scheme 2). In addition, 2,4-disubstituted oxazoles can be prepared from α-haloketones and amides at higher temperatures using the same method.

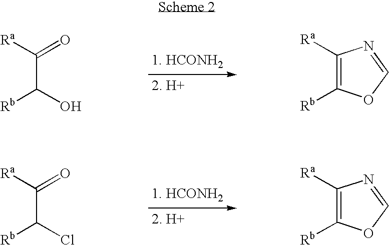

Oxazoles can be prepared by cyclization reactions of isonitriles (van Leusen, A. M. Lect. Heterocycl. Chem. 1980, 5, S111; Walborsky, H. M.; Periasamy, M. P. in The Chemistry of Functional Groups, suppl. C, Patai, S.; Rappoport, Z., Eds; Wiley-Interscience, 1983, p. 835; Hoppe, D. Angew. Chem. Int. Edn. Engl., 1974, 13, 789; Schollkopf, U. Angew. Chem. Int. Ed. Engl., 1977, 16, 339). For example, as shown below in Scheme 3, the tosylmethyl isocyanide can be deprotonated by a base and reacted with a suitable electrophile (e.g. an aldehyde). The intermediate can cyclize and aromatize to provide the desired oxazole analog. Other methods for preparing oxazoles include 1,5-dipolar cyclization of acylated nitrile ylides (Taylor E. C.; Turchi, I. J. Chem. Rev., 1979, 79, 181; Huisgen, R. Angew. Chem. Int. Edn. Engl. 1980, 19, 947)

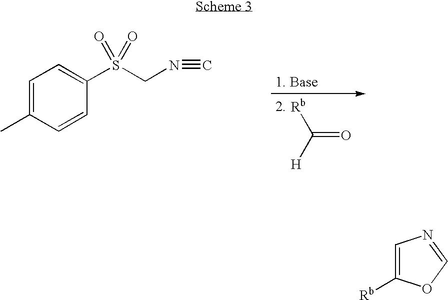

2-Amino-substituted oxazoles (i.e. R^{c}═NH₂) can be prepared by two general methods. Urea can be condensed with α-bromo ketones to yield 2-aminooxazoles that can be substituted at the 4 and 5 positions (Scheme 4). Alternatively, another route to 2-aminooxazoles from acyclic precursors is the base catalyzed reaction of cyanamide with α-hydroxy ketones (Scheme 5). 2-Aminooxazoles of the invention can also be prepared from the nucleophilic displacement of amines with 2-chlorooxazole, for example, for compounds of the invention wherein R^{c} is ArNH— (Gompper, R.; Effenberger, F. Chem. Ber., 1959, 92, 1928).

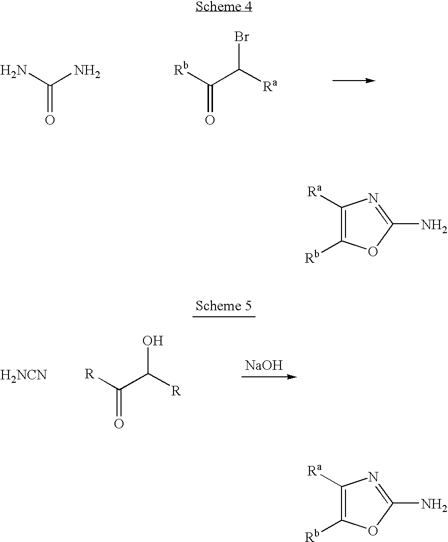

Compounds of the invention, wherein R^{c} is arylcarbonyl can be synthesized by acylation of the amino moiety of 2-aminooxazoles with, for example, with anhydrides to yield 2-acylaminooxazoles. In addition, 2-aminooxazoles can be acylated, for instance, with chloroacetic anhydride to yield an α-chloro carboxamide. The α-chloro carboxamide can serve as a suitable alkylating reagent that can be treated with, for example, phenols, arylamines and alkylamines to prepare compounds of the invention. An oxazole of the invention wherein R^{c} is Ar-oxycarbonylamino is shown in Scheme 6.

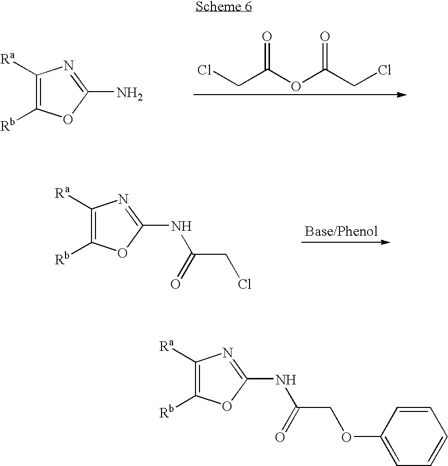

Oxazoles of the invention with R^{c} is aminocarbonylamino (ureido) or aminothiocarbonylamino (thioureido) can be prepared from 2-aminooxazoles (Scheme 7). 2-Aminooxazoles can be treated with isocyanates and isothiocyanates to yield 2-ureido and 2-thioureido oxazoles, respectively (Crank, G.; Foulis, J. J. Med. Chem., 1971, 14, 1075: Crank, G. Neville, M.; Ryden, R. J. Med. Chem., 1974, 16, 1402).

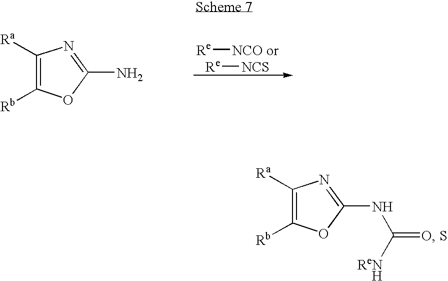

2-Aminooxazoles can be hydrogenated using palladium catalysts to yield 2-aminooxazolines (Scheme 8) (Tanaka, C.; Kuriyama, S. Yakugaku Zasshi 1979, 99, 78).

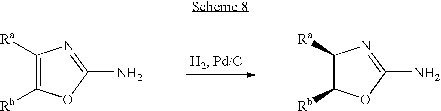

Benzoxazole intermediates substituted at the 2 position can be prepared from 2-aminophenols by acylation with, for example, with an acid chloride and cyclization (Scheme 9).

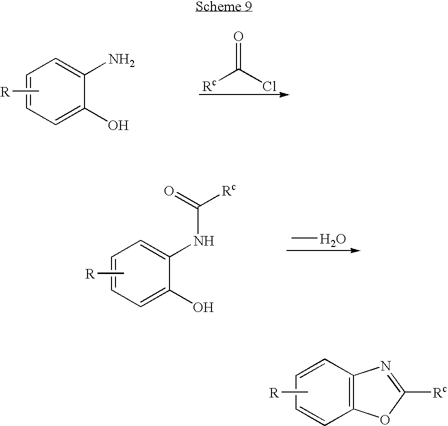

To treat glaucoma or reduced accommodation and associated symptoms, an effective amount of a pharmaceutical compound will be recognized by clinicians but includes an amount effective to treat, reduce, ameliorate, eliminate or prevent one or more symptoms of the disease sought to be treated or the condition sought to be avoided or treated, or to otherwise produce a clinically recognizable change in the pathology of the disease or condition.

In treating glaucoma, agents of the inventions can be administered concurrently or in a combined formulation with one or more α₂-selective adrenergic agonists, carbonic anhydrase inhibitors or prostaglandin analogs. Examples of α₂-selective adrenergic agonists include clonidine, apraclonidine, guanfacine, guanabenz and methyldopa, which are administered in effective amounts as is known in the art. Examples of carbonic anhydrase inhibitors include acetazolamide, dichlorphenamide and methazolamide, which are administered in effective amounts as is known in the art. Examples of prostaglandin analogs include PGE₂ and PGF_{2α} analogs, which are administered in effective amounts as is known in the art, including effective amounts administered by topical application to the eye. Thus, the invention further provides pharmaceutical compositions comprising an agent of the invention in combination with an effective amount of an α₂-selective adrenergic agonist, carbonic anhydrase inhibitor, prostaglandin analog, or combination thereof.

Pharmaceutical compositions can be prepared to allow a therapeutically effective quantity of the compound of the present invention, and can include a pharmaceutically acceptable carrier, selected from known materials utilized for this purpose. See, e.g., Remington, The Science and Practice of Pharmacy, 1995; Handbook of Pharmaceutical Excipients, 3^{rd} Edition, 1999. Such compositions can be prepared in a variety of forms, depending on the method of administration.

In addition to the subject compound, the compositions of this invention can contain a pharmaceutically-acceptable carrier. The term “pharmaceutically-acceptable carrier”, as used herein, means one or more compatible solid or liquid filler diluents or encapsulating substances that are suitable for administration to an animal, including a mammal or human. The term “compatible”, as used herein, means that the components of the composition are capable of being commingled with the subject compound, and with each other, such that there is no interaction that would substantially reduce the pharmaceutical efficacy of the composition under ordinary use. Preferably when liquid dose forms are used, the compounds of the invention are soluble in the components of the composition. Pharmaceutically-acceptable carriers must, of course, be of sufficiently high purity and sufficiently low toxicity to render them suitable for administration to the animal being treated.

Some examples of substances which can serve as pharmaceutically-acceptable carriers or components thereof are sugars, such as lactose, glucose and sucrose; starches, such as corn starch and-potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject compound is basically determined by the way the compound is to be administered. If the subject compound is to be injected, the preferred pharmaceutically-acceptable carrier is sterile, physiological saline, with a blood-compatible suspending agent, the pH of which has been adjusted to about 7.4.

If the preferred mode of administering the subject compound is perorally, the preferred unit dosage form is therefore tablets, capsules, lozenges, chewable tablets, and the like. Such unit dosage forms comprise a safe and effective amount of the subject compound, which is preferably from about 0.7 or 3.5 mg to about 280 mg/70 kg, more preferably from about 0.5 or 10 mg to about 210 mg/70 kg. The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder-mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of this invention, and can be readily made by a person skilled in the art.

Peroral compositions also include liquid solutions, emulsions, suspensions, and the like. The pharmaceutically-acceptable carriers suitable for preparation of such compositions are well known in the art. Such liquid oral compositions preferably comprise from about 0.012% to about 0.933% of the subject compound, more preferably from about 0.033% to about 0.7%. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. Peroral liquid compositions may also contain one or more components such as sweeteners, flavoring agents and colorants disclosed above.

Other compositions useful for attaining systemic delivery of the subject compounds include sublingual and buccal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Compositions can also be used to deliver the compound to the site where activity is desired; such as eye drops, gels and creams for ocular disorders.

Compositions of this invention include solutions or emulsions, preferably aqueous solutions or emulsions comprising a safe and effective amount of a subject compound intended for topical intranasal administration. Such compositions preferably comprise from about 0.01% to about 10.0% w/v of a subject compound, more preferably from about 0.1% to about 2.0%. Similar compositions are preferred for systemic delivery of subject compounds by the intranasal route. Compositions intended to deliver the compound systemically by intranasal dosing preferably comprise similar amounts of a subject compound as are determined to be safe and effective by peroral or parenteral administration. Such compositions used for intranasal dosing also typically include safe and effective amounts of preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acids and bases to adjust the pH of these aqueous compositions as needed. The compositions may also comprise local anesthetics or other actives. These compositions can be used as sprays, mists, drops, and the like.

Other preferred compositions of this invention include aqueous solutions, suspensions, and dry powders comprising a safe and effective amount of a subject compound intended for atomization and inhalation administration. Such compositions are typically contained in a container with attached atomizing means. Such compositions also typically include propellants such as chlorofluorocarbons 12/11 and 12/114, and more environmentally friendly fluorocarbons, or other nontoxic volatiles; solvents such as water, glycerol and ethanol, these include cosolvents as needed to solvate or suspend the active; stabilizers such as ascorbic acid, sodium metabisulfite; preservatives such as cetylpyridinium chloride and benzalkonium chloride; tonicity adjustors such as sodium chloride; buffers; and flavoring agents such as sodium saccharin. Such compositions are useful for treating respiratory disorders, such as asthma and the like.

Other preferred compositions of this invention include aqueous solutions comprising a safe and effective amount of a subject compound intended for topical intraocular administration. Such compositions preferably comprise from about 0.01% to about 0.8% w/v of a subject compound, more preferably from about 0.05% to about 0.3%. Such compositions also typically include one or more of preservatives, such as benzalkonium chloride or thimerosal; vehicles, such as poloxamers, modified celluloses, povidone and purified water; tonicity adjustors, such as sodium chloride, mannitol and glycerin; buffers such as acetate, citrate, phosphate and borate; antioxidants such as sodium metabisulfite, butylated hydroxy toluene and acetyl cysteine; acids and bases can be used to adjust the pH of these formulations as needed.

Other preferred compositions of this invention useful for peroral administration include solids, such as tablets and capsules, and liquids, such as solutions, suspensions and emulsions (preferably in soft gelatin capsules), comprising a safe and effective amount of a subject compound. Such compositions can be coated by conventional methods, typically with pH or time-dependent coatings, such that the subject compound is released in the gastrointestinal tract at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

The compounds of the invention are administered by ocular, oral, parenteral, including, for example, using formulations suitable as eye drops. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. See, Remington's Pharmaceutical Sciences, 16th Ed., Mack Publishing, Easton, Pa., 1980, as well as later editions, for information on pharmaceutical compounding.

Numerous additional administration vehicles will be apparent to those of ordinary skill in the art, including without limitation slow release formulations, liposomal formulations and polymeric matrices.

In another preferred embodiment, the pharmaceutically effective amount is approximately 0.1 or 0.5 to 4 mg/kg body weight daily. Still more preferably, the pharmaceutically effective amount is approximately 1 mg/kg body weight daily. In a preferred embodiment, the amount is administered in once daily doses, each dose being approximately 1 mg/kg body weight.

Compounds of the invention can be used in conjunction with monitoring the improvement (decrease) in the intraocular pressure in a mammal using standard methodology.

The methods of the inventions can be assessed in animal models for ophthalmologic function. For example, improvements in fluid outflow facility can be studied in Rhesus monkeys treated with the compounds and methods of the invention. Aged Rhesus monkeys receive a single transcorneal injection of a test compound (compound of the invention) at a concentration of about 1 mM in the anterior chamber of one eye, and Barany's solution, as a control, in the adjacent eye. Needle outflow facility is measured under baseline and pilocarpine-stimulated conditions at time points (for example, 3, 8, 12 and 24 weeks), after the administration of the test compound. Increases in outflow facility in the drug treated vs. the control eye under baseline and cholinergic-stimulated (e.g. pilocarpine) conditions at the various time points are compared. As the enhancement of outflow facility can be influenced by the route of administration of the cholinergic agent, various routes of administration of the cholinergic agent can be used in the experiments. For instance, an intravenous administration versus a direct administration of pilocarpine can be compared. The above experiment demonstrates one method of measuring the improvement in ophthalmologic function. Such improvement has been illustrated with 4,5-dimethyl-3-(2-oxoethyl-phenethyl)thiazolium chloride, a compound believed to act by the same mechanism as those described here. See, U.S. application for “Methods for Treating Glaucoma I,” concurrently filed herewith.

In addition to measuring increased fluid outflow facility using the methods of the invention, improvements in pilocarpine-stimulated accommodation (i.e, the process of effecting refractive changes in the shape of the lens) can also be assessed in animal studies. As in the regulation of outflow facility, cholinergic input stimulates the movement of the ciliary muscle to control the shape of the lens, and allows accommodation in conditions of low illumination. Accommodation is impaired in a vast majority of individuals and begins to become noticeable to the individual around the age of 40 years. Interestingly, changes in accommodative response occur much earlier in life, around 18 years of age, and progresses until vision is noticeably impaired.

Physiological studies on accommodation are conducted following intraocular injection of a test compound and the results are compared relative to the results of control (untreated) animals. In the experiment, primates (for example, Rhesus monkeys) are treated twice a day for four days with 2 μg of prostaglandin F2α (PGF2α). On days 5-8 both eyes are treated first with 2 μg of PGF2α followed 2 hours later with an intraocular injection of 10 μL of the test compound of a final concentration of 1 mM. No injection is made to the control eye. 24 hours after the last injection of the test compound, a course of therapy consisting of once a day dosing for a total of 4 days accommodative responses to i.m. pilocarpine administration is performed following phenylephrine refraction. Improvement in accommodation has been illustrated with 4,5-dimethyl-3-(2-oxoethyl-phenethyl)thiazolium chloride, a compound believed to act by the same mechanism as those described here. See, U.S. application for “Methods for Treating Glaucoma I,” concurrently filed herewith.

Compounds of the invention can be tested to determine corneal penetration to the anterior chamber of the eye following topical administration of eye drops. For example, a test compound is assayed in vitro through an intact rabbit cornea for transcorneal penetration in a standard diffusion chamber apparatus. Corneas are mounted in a chamber at 37° C. with the epithelial side exposed to the test compound in Barany's solution. 1.0 mL samples are taken from the endothelial side 1 hour after addition of the test compound at a final concentration of 1 mM to the epithelial chamber. The volume of the chamber is replaced with phosphate buffered saline. The amount of test compound can be measured using any means that can be used to separate the compound and measure its concentration. For example, an HPLC with an attached UV detector can be used to determine the concentration of the test compound that has penetrated the cornea. Penetration values are also determined at later time points, for example, at 5 hours.

Assessment of corneal penetration of compounds of the invention can be determined in vivo, for example, in Cynomolgus monkeys. During these studies, the penetration of a test compound is evaluated using an eye-cup which holds a solution of 10 mM of the test compound in Barany's solution for 5 hours. At the end of the experiment the eye cup is removed, the eye is repeatedly flooded with Barany's solution and a sample of intraocular fluid is removed from the anterior chamber with a needle inserted through the cornea. The quantity of the test compound in the intraocular fluid is determined using, for example, HPLC methods.

The activity of the compounds of the invention in breaking, reversing or inhibiting the formation of AGE's or AGE-mediated cross-links can be assayed by any of the methods described in U.S. Pat. No. 5,853,703.

The following examples further illustrate the present invention, but of course, should not be construed as in any way limiting its scope.

### EXAMPLE 1

2,6-diamino-benzothiazole dihydrochloride: 4 g of 2-amino-6-nitrobenzothiazole (Aldrich) was suspended in 130 ml MeOH, and 0.4 g 10% Pd/c (Aldrich) added. The suspension was hydrogenated at room temperature under 60 psi H₂ for 6.5 h. The reaction mixture was filtered, and the particulate washed with MeOH. The filtrate was concentrated under reduced pressure, and crystals formed from the concentrate were collected to yield 2.67 g. mp 196-198° C., yield 81.6%. 0.91 g of this product was dissolved in 22 ml MeOH, and the pH adjusted with HCl to 4 to produce 1.2 g of crystals of 2,6-diamino-benzothiazole dihydrochloride. mp 318-320° C., 92.3% yield. Anal. calc. for C₇H₉N₃SCl₂, C, 35.30%; H, 3.80%; N, 17.64%. Found, C, 34.91%; H, 3.67%; N, 17.71%.

### EXAMPLE 2

2-(3,5-Dimethylphenoxy)-N-thiazol-2-yl)acetamide: First Route: 3,5-Dimethylphenol is reacted with bromoacetic acid at 110° C. for four hours, with the reaction mixture stirred overnight without added heat. The resulting (3,5-dimethylphenoxy)acetic acid is dissolved in methylene chloride and coupled to 2-aminothiazole in an overnight, room temperature reaction conducted in the presence of base (N-methylmorpholine) and dehydration mediators 1-hydroxybenzotriazole and 1-(3-dimethylaminopropyl)-3-ethyl-carbodiimide hydrochloride.

Second Route: 3,5-Dimethylphenol is reacted for 4.5 h with bromoacetic acid in THF under nitrogen and in the presence of sodium hydride. The resulting (3,5-dimethylphenoxy)acetic acid is reacted overnight with thionyl chloride, with heat. The resulting (3,5-dimethylphenoxy)acetyl chloride is reacted overnight with 2-aminothiazole in the presence of triethylamine, with cooling to 0° C.

Third Route: 2-Aminothiazole (20 g, 199.7 mmol) was suspended in methylene chloride (200 ml), in the presence of pyridine (20 ml, ˜250 mmol), and the mixture cooled to 0° C. Bromoacetyl bromide (18.1 ml, 207.6 mmol) was dissolved in 400 ml methylene chloride, and this solution added to the suspended 2-aminothiazole dropwise. The resulting reaction mixture was stirred at room temperature overnight. The crude product was washed with water (200 ml, 1×), then sodium bicarbonate solution (200 ml, 2×), dried over Na₂SO₄, filtered, and evaporated. The product 2-bromoacetamidothiazole was crystallized from MeOH. Yield, 4 g. mp 148° C.

A solution of 3,5-dimethylphenol (2.5 g, 13.9 mmol) in dry DMF (20 ml) was placed under a dry nitrogen atmosphere. Sodium hydride (0.7 g, 27.8 mmol; a 60% dispersion in mineral oil) was added in portions, and the mixture stirred for 1 h. A solution of 2-bromoacetamidothiazole (3.0 g, 13.9 mmol) in dry DMF (10 ml) was added to the mixture dropwise. The reaction was heated to 90° C. for 5 h, then maintained overnight without external heat. The reaction mixture was poured into ice water, and the resulting material extracted with methylene chloride (50 ml×3). The organic layer was washed with water (100 ml×5), dried over Na₂SO₄, filtered, and evaporated. The residue from evaporation was purified by silica gel chromatography developed with pet. ether:ether (1:1 v/v). The product N-(Thiazolyl)-2-(3,5-dimethylphenoxy)-acetamide was crystallized from acetonitrile and methyl tert-butyl ether. Yield, 1.04 g. mp 124-125° C.

### EXAMPLE 3

2-Furyl-N-[4-(6-methyl-benzothiazol-2-yl)phenyl]carboxamide: First Route: 2-Furoic acid (1.85 gms, 16.5 mmole) was dissolved in anhydrous methylene chloride (30 mls), to which solution was added a suspension of 2-(4-amino-phenyl)-6-methyl benzothiazole (4.76 g., 16.5 mmole) and N-methyl morpholine (2.0 g., 16.5 mmole) in methylene chloride (30 mls, at room temperature). Then, 1-hydroxy-benzotriazole hydrate (2.67 g., 16.5 mmole) and 1-(3-dimethyl amino propyl)-3-ethyl carbodiimide hydrochloride (4.75 g., 16.5 mmole) were added at room temperature. More methylene chloride (20 ml.) was added with stirring at room temperature, and the reaction maintained overnight. The initial clear reaction solution changed to a turbid solution. More methylene chloride (10 ml.) was added to the product mixture, which was then extracted with 1N HCl to separate a solid. The solid was filtered and washed with water. The product solid was crystallized from large amount of MeOH to yield 2.19 gm. (33.1%). mp. 238-240° C. ¹H and ¹³C NMR were consistent with the expected product. TLC showed one spot (5% MeOH—CH₂Cl₂ as developing solvent on silica gel plate).

Route 2: 2-(4-aminophenyl)-6-methyl benzothiazole (2.0 gm, 8.3 mmole) and 2-Furoyl chloride (1.086 gm., 8.32 mmole) were suspended in methylene chloride (30 ml, anhydrous). triethylamine (1.24 gm., 12.25 mmole) was added to the reaction mixture with stirring at room temperature for 2 days. (pH 7.0-7.2). Methylene chloride (50 ml) was added to the reaction mixture, and the reaction mixture extracted with 1 N HCl (50 ml) to separate a solid. The solid was filtered and washed with water to yield 1.3 gm. (46%) of the desired compound. The product was crystallized from MeOH to obtain 0.99 gm. mp. 238-240° C. ¹H and ¹³C NMR were consistent with the expected product. TLC showed one spot (5% MeOH—CH₂Cl₂ as developing solvent on silica gel plate).

### EXAMPLE 4

### Cross-Linking Inhibition Assay

The following method was used to evaluate the ability of the compounds to inhibit the cross-linking of glycated bovine serum albumin (AGE-BSA) to rat tail tendon collagen-coated 96-well plates.

AGE-BSA was prepared by incubating BSA at a concentration of 200 mg per ml with 200 mM glucose in 0.4M sodium phosphate buffer, pH 7.4 at 37° C. for 12 weeks. The glycated BSA was then extensively dialyzed against phosphate buffer solution (PBS) for 48 hours with additional 5 times buffer exchanges. The rat tail tendon collagen coated plate was blocked first with 300 microliters of Superbloc blocking buffer (Pierce Chemical, Rockford, Ill.) for one hour. The blocking solution was removed from the wells by washing the plate twice with phosphate buffered saline (PBS)-Tween 20 solution (0.05% Tween 20) using a NUNC-multiprobe (Nalge Nunc, Rochester, N.Y.) or Dynatech ELISA-plate (Dynatech, Alexandria, Va.) washer. Cross-linking of AGE-BSA (1 to 10 microgram per well depending on the batch of AGE-BSA) to rat tail tendon collagen coated plate was performed with and without the testing compound dissolved in PBS buffer at pH 7.4 at one or more desired concentrations by the addition of 50 microliters each of the AGE-BSA diluted in PBS or in the solution of test compound at 37° C. for 4 hours. Unbrowned BSA in PBS buffer with or without testing compound were added to the separate wells as the blanks. The un-cross-linked AGE-BSA was then removed by washing the wells three times with PBS-Tween buffer. The amount of AGE-BSA crosslinked to the tail tendon collagen-coated plate was then quantitated using a polyclonal antibody raised against AGE-RNase. After a one-hour incubation period, AGE antibody was removed by washing 4 times with PBS-Tween.

The bound AGE antibody was then detected with the addition of horseradish peroxidase-conjugated secondary antibody-goat anti-rabbit immunoglobulin and incubation for 30 minutes. The substrate of 2,2-azino-di(3-ethylbenzthiazoline sulfonic acid) (ABTS chromogen) (Zymed Laboratories, Inc., South San Francisco, Calif.) was added. The reaction was allowed for an additional 15 minutes and the absorbance was read at 410 nm in a Dynatech plate reader.

### EXAMPLE 5 CROSS-LINK BREAKING ASSAY

To ascertain the ability of the compounds of the instant invention to break or reverse already formed advanced glycosylation endproducts, a sandwich enzyme immunoassay was applied. Generally, the assay utilizes collagen-coated 96 well microtiter plates that are obtained commercially. AGE-modified protein (AGE-BSA) is incubated on the collagen-coated wells for four hours, is washed off the wells with PBS-Tween and solutions of the test compounds are added. Following an incubation period of 16 hours (37° C.) cross-link-breaking is detected using an antibody raised against AGE-ribonuclease or with an antibody against BSA.

Preparation of Solutions and Buffers

Bovine Serum Albumin (Type V) (BSA) (from Calbiochem) solution was prepared as follows: 400 mg of Type V BSA (bovine serum albumin) was added for each ml of 0.4 M sodium phosphate buffer, pH 7.4. A 400 mM glucose solution was prepared by dissolving 7.2 grams of dextrose in 100 ml of 0.4 M sodium phosphate buffer, pH 7.4. The BSA and glucose solutions were mixed 1:1 and incubated at 37° C. for 12 weeks. The pH of the incubation mixture was monitored weekly and adjusted to pH 7.4 if necessary. After 12 weeks, the AGE-BSA solution was dialyzed against PBS for 48 hours with four buffer changes, each at a 1:500 ratio of solution to dialysis buffer. Protein concentration was determined by the micro-Lowry method. The AGE-BSA stock solution was aliquoted and stored at −20° C.

Test compounds were dissolved in PBS and the pH was adjusted to pH 7.4, if necessary. AGE-BSA stock solution was diluted in PBS to measure maximum crosslinking and in the inhibitor solution for testing inhibitory activity of compounds. The concentration of AGE-BSA necessary to achieve the optimum sensitivity was determined by initial titration of each lot of AGE-BSA.

Substrates for detection of secondary antibody binding were prepared by diluting the HRP substrate buffer (Zymed) 1:10 in distilled water and mixing with ABTS chromogen (Zymed) 1:50 just prior to use.

Assay Procedures

Biocoat plates were blocked with 300 microliters of Superbloc (Pierce Chemical). Plates were blocked for one hour at room temperature and were washed with PBS-Tween (0.05% v/v) three times with the Dynatech platewasher before addition of test reagents.

The first three wells of the Biocoat plate were used for the reagent blank. Fifty microliters of solutions AGE-BSA were added to test wells in triplicate and only PBS in blank wells. The plate was incubated at 37° C. for four hours and washed with PBS-Tween three times. Fifty microliters of PBS was added to the control wells and 50 microliters of the test prospective agent was added to the test wells and blank. The plate was incubated overnight (approximately 16 hours) with prospective agent, followed by washing in PBS before addition of primary antibody.

(Prior to use, each lot of primary antibody, either anti-BSA or anti-RNase, was tested for optimum binding capacity in this assay by preparing serial dilutions (1:500 to 1:2000) and plating 50 microliters of each dilution in the wells of Biocoat plates. Optimum primary antibody was determined from saturation kinetics.) Fifty microliters of primary antibody of appropriate dilution, was added and incubated for one hour at room temperature. The plate was then washed with PBS-Tween.

Plates were incubated with the secondary antibody, HRP-(Goat-anti-rabbit), which was diluted 1:4000 in PBS and used as the final secondary antibody. The incubation was performed at room temperature for thirty minutes.

Detection of maximum crosslinking and breaking of AGE crosslinking was performed as follows. HRP substrate (100 microliter) was added to each well of the plate and was incubated at 37° C. for fifteen minutes. Readings were taken in the Dynatech ELISA-plate reader.

Definitions

Heterocycle. Except where heteroaryl is separately recited for the same substituent, the term “heterocycle” includes heteroaryl.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

1. A method of decreasing intraocular pressure or improving ocular accommodation in an animal, including a human, comprising administering an intraocular pressure decreasing amount or ocular accommodation improving amount of a compound of the formula I or IA, 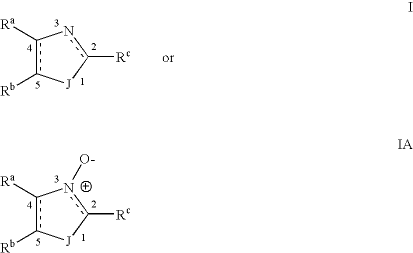
wherein:
a. J is oxygen, sulfur, or N—R^{d};
b. the carbon 2 to nitrogen bond is a double bond except when R^{c} is oxo;
c. the bond between carbons 4 and 5 is a single bond or a double bond;
d. R^{a} and R^{b} are
1. independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, co-alkylenesulfonic acid, carbamoyl, carboxy, carboxyalkyl, which alkyl can be substituted with alkyloxyimino, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar, wherein, consistent with the rules of aromaticity, Ar is C₆ or C₁₀ aryl or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring can be fused to a substituted benzene, pyridine, pyrimidine, pyrazine, pyridazine, or (1,2,3)triazine, wherein the ring fusion is at a carbon-carbon double bond of Ar, Ar-alkyl, ArO—, ArSO₂—, ArSO—, ArS—, ArSO₂NH—, ArNH, (N—Ar)(N-alkyl)N—, ArC(O)—, ArC(O)NH—, ArNH—C(O)—, and (N—Ar)(N-alkyl)N—C(O)—, or together R^{a} and R^{b} comprise methylenedioxy-; or
2. together with their ring carbons form a C₆- or C₁₀-aryl fused ring; or
3. together with their ring carbons form a C₅-C₇ fused cycloalkyl ring having up to two double bonds including a fused double bond of the containing group, which cycloalkyl ring can be substituted by one or more of the group consisting of alkyl, alkoxycarbonyl, amino, aminocarbonyl, carboxy, fluoro, or oxo; or
4. together with their ring carbons form a fused 5- or 6-membered heteroaryl ring, wherein the 6-membered heteroaryl ring contains one to three atoms of N, and the 5-membered heteroaryl ring contains from one to three atoms of N or one atom O or S and zero to two atoms of N; or
5. together with their ring carbons form a fused five to eight membered second heterocycle, wherein the fused heterocycle consists of ring atoms selected from the group consisting of carbon, nitrogen, oxygen, sulfur, and S(O)ₙ, wherein n is 1 or 2;
e. R^{d} is alkyl, alkenyl, hydrogen, or Ar;
f. R^{c} is
1. oxo when Δ^{2,3} is not present, or, when Δ^{2,3} is present, hydrogen, alkyl, alkylthio, hydrogen, mercapto, amino, amino(C₁-C₅)alkyl, amino(C₆ or C₁₀)aryl, or wherein the amino of the last three groups can be substituted with
(a) Ar,
(b) Ar-Z-, Ar-alkyl-Z-, Ar-Z-alkyl, Ar-amino-Z-, Ar-aminoalkyl-Z-, or Ar-oxyalkyl-Z-, wherein Z is a carbonyl or —SO₂—,
(c) formyl or alkanoyl,
2. —NHC(O)(CH₂)ₙ-D-R^{e}R^{f}, wherein D is oxygen, sulfur or nitrogen, wherein where D is nitrogen n is 0, 1 or 2, but when D is oxygen or sulfur n=1 or 2, and R^{f} is present only when D is nitrogen,
wherein
(a) R^{e} is
(1) Ar,
(2) a group of the formula II, 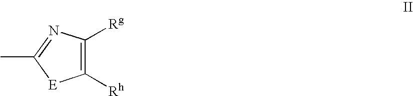
wherein E is sulfur, oxygen, or N—Rⁱ, and R^{g}, R^{h} and Rⁱ are independently the same as R^{a}, R^{b} and R^{d}, respectively,
(3) a C₃-C₈ cycloalkyl ring having up to one double bond with the proviso that the carbon linking the cyloalkyl ring to D is saturated, which cycloalkyl ring can be substituted by one or more alkyl-, alkoxycarbonyl-, amino-, aminocarbonyl-, carboxy-, fluoro-, or oxo-substituents;
(4) a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings;
(5) hydrogen, (C₂-C₆)hydroxyalkyl, alkanoylalkyl, alkyl, alkoxycarbonylalkyl, alkenyl, carboxyalkyl, which alkyl can be substituted with alkoxyimino, alkoxycarbonyl, a group Ar^{Φ} which is C₆- or C₁₀-aryl or a 5- or 6-membered, or 9- or 10-membered heteroaryl, wherein the heteroatom is one oxygen, one sulfur or one nitrogen, or Ar^{Φ}-alkyl; and
(b) R^{f} is independently hydrogen, (C₂-C₆)hydroxyalkyl, alkanoylalkyl, alkyl, alkoxycarbonylalkyl, alkenyl, carboxyalkyl, which alkyl can be substituted with alkyloxyimino, alkoxycarbonyl, Ar^{Φ}, or Ar^{Φ}-alkyl;
wherein aryl, Ar, or Ar^{Φ}) can be substituted with, in addition to any substitutions specifically
noted one or more substituents selected from the group of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, co-alkylenesulfonic acid, alkylthio, allyl, amino, ArC(O)—, ArC(O)NH—, carboxy, carboxyalkyl, cycloalkyl, dialkylamino, halo, trifluoromethyl, hydroxy, (C₂-C₆)hydroxyalkyl, mercapto, nitro, ArO—, Ar—, Ar-alkyl-, sulfamoyl, sulfonic acid, 1-pyrrolidinyl, 4-[C₆ or C₁₀]arylpiperazin-1-yl-, 4-[C₆ or C₁₀]arylpiperidin-1-yl, azetidin-1-yl, morpholin-4-yl, thiomorpholin-4-yl, piperazin-1-yl, piperidin-1-yl; and
heterocycles, except those of Ar and Ar^{Φ}, can be substituted with in addition to any substitutions specifically noted one or more substituents selected from acylamino, alkanoyl, alkoxy, alkoxycarbonyl, alkoxycarbonylalkyl, alkyl, (C₁ to C₃)alkylenedioxy, alkylamino, alkylsulfonyl, alkylsulfinyl, alkylthio, amino, ArC(O)—, ArO—, Ar—, Ar-alkyl, carboxy, dialkylamino, fluoro, fluoroalkyl, difluoroalkyl, hydroxy, mercapto, oxo, sulfamoyl, trifluoromethyl, 4-[C₆ or C₁₀]arylpiperidin-1-yl and 4-[C₆ or C₁₀]arylpiperazin-1-yl-;
or a pharmaceutically acceptable salt of said compound.

2. The method of claim 1, comprising administering an intraocular pressure decreasing amount or ocular accommodation improving amount of a compound of the formula I, wherein the bond between carbons 4 and 5 is a single bond.

3. The method of claim 1, comprising administering an intraocular pressure decreasing amount or ocular accommodation improving amount of a compound of the formula I, wherein R^{c} is amino, amino(C₁-C₅)alkyl, or amino(C₆ or C₁₀)aryl, or wherein the amino of any of the three groups can be substituted with
(a) Ar;
(b) Ar-Z-, Ar-alkyl-Z-, Ar-Z-alkyl, Ar-amino-Z-, Ar-aminoalkyl-Z-, or Ar-oxyalkyl-Z-; or
(c) formyl or alkanoyl.

4. The method of claim 1, comprising administering an intraocular pressure decreasing amount or ocular accommodation improving amount of a compound of the formula I, wherein J is S or O, and R^{c} is hydrogen, oxo, alkyl, amino, amino(C₁-C₅)alkyl or aminophenyl, wherein the amino of the latter three groups can be substituted with
(a) Ar;
(b) Ar-Z-, Ar-alkyl-Z-, Ar-Z-alkyl, Ar-amino-Z-, Ar-aminoalkyl-Z-, or Ar-oxyalkyl-Z-; or
(c) formyl or alkanoyl.

5. The method of claim 1, administering an intraocular pressure decreasing amount or ocular accommodation improving amount of a compound of the formula I, wherein J is S, and R^{c} is hydrogen, oxo, alkyl, amino, amino(C₁-C₅)alkyl or aminophenyl, wherein the amino of the latter three groups can be substituted with
(a) Ar;
(b) Ar-Z-, Ar-alkyl-Z-, Ar-Z-alkyl, Ar-amino-Z-, Ar-aminoalkyl-Z-, or Ar-oxyalkyl-Z-; or
(c) formyl or alkanoyl.

6. The method of claim 1, administering an intraocular pressure decreasing amount or ocular accommodation improving amount of a compound of the formula I, wherein the compound is selected from the group consisting of thiazole, 2-amino-4-chlorobenzothiazole, 2,4,5-trimethylthiazole, 2-(3,5-dimethylphenoxy)-N-thiazol-2-yl)acetamide-, 2-isobutylthiazole, (4-fluorophenyl)thiazolin-2-ylamine, 2-furyl-N-[4-(6-methylbenzothiazol-2-yl)phenyl]carboxamide, and 5,5-dimethyl-2-(2-naphthylamino)-4,5,6-trihydrobenzothiazol-7-one.

7. The method of claim 1, comprising administering an intraocular pressure decreasing amount or ocular accommodation improving amount of a compound of the formula I, wherein
d. R^{a} and R^{b} are
1. independently selected from hydrogen, acylamino, alkanoyl, alkanoylalkyl, alkoxy, alkoxycarbonyl, alkoxycarbonylalkyl, alkyl, alkylamino, amino, ω-alkylenesulfonic acid, carbamoyl, carboxy, carboxyalkyl, which alkyl can be substituted with alkyloxyimino, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar, wherein, consistent with the rules of aromaticity, Ar is C₆ or C₁₀ aryl or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring can be fused to a substituted benzene, pyridine, pyrimidine, pyridazine, or (1,2,3)triazine, wherein the ring fusion is at a carbon-carbon double bond of Ar, Ar-alkyl, ArO—, ArSO₂—, ArSO—, ArS—, ArSO₂NH—, ArNH, (N—Ar)(N-alkyl)N—, ArC(O)—, ArC(O)NH—, ArNH—C(O)—, and (N—Ar)(N-alkyl)N—C(O)—; or
2. together with their ring carbons form a C₆- or C₁₀-aryl fused ring; or
3. together with their ring carbons form a C₅-C₇ fused cycloalkyl ring having no double bonds except a fused double bond of the formula I or IA ring, which cycloalkyl ring can be substituted by one or more of the group consisting of alkyl, amino, aminocarbonyl, carboxy, fluoro, or oxo, where multiple substituents are located on different carbon atoms of the cycloalkyl ring, except in the case of alkyl and fluoro substituents, which can be located on the same or different carbon atoms; or
4. together with their ring carbons form a fused 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N; or
5. together with their ring carbons form a fused five to six membered second heterocycle, wherein the fused heterocycle consists of ring atoms selected from the group consisting of carbon, nitrogen, oxygen, sulfur, and S(O)ₙ, wherein n is 1 or 2,
wherein aryl, Ar, or Ar^{Φ} can be substituted with, in addition to any substitutions specifically noted one or more substituents selected from the group of alkyl, amino, dialkylamino, 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperazin-1-yl, piperidin-1-yl; and
heterocycles, except those of Ar and Ar^{Φ}, can be substituted with in addition to any substitutions specifically noted one or more substituents selected from acylamino, alkanoyl, alkoxy, alkoxycarbonyl, alkoxycarbonylalkyl, alkyl, (C₁ or C₃)alkylenedioxy, alkylamino, alkylsulfonyl, alkylsulfinyl, alkylthio, amino, ArC(O)—, ArO—, Ar—, Ar-alkyl, carboxy, dialkylamino, fluoro, fluoroalkyl, difluoroalkyl, hydroxy, mercapto, oxo, sulfamoyl, trifluoromethyl, 4-[C₆ or C₁₀]arylpiperidin-1-yl and 4-[C₆ or C₁₀]arylpiperazin-1-yl, wherein multiple substituents are located on different atoms of the heterocyclic ring, with the proviso that alkyl, alkoxycarbonyl, and fluoro substituents can be substituted on the same carbon atom of the heterocyclic ring.
